(19) United States
(12) Patent Application Publication
(10) Pub. No.: US 2006/0198899 A1
(43) Pub. Date: Sep. 7, 2006

(54) Supplemental dietary composition for supporting muscle growth, recovery and strength

(76) Inventors: Paul T. Gardiner, Mississauga (CA); Marvin A. Heuer, Mississauga (CA)

Correspondence Address: KENYON & KENYON LLP, ONE BROADWAY, NEW YORK, NY, 10004, US

(21) Appl. No.: 11/361,539

(22) Filed: Feb. 24, 2006

Related U.S. Application Data
(60) Provisional application No. 60/657,680, filed on Mar. 1, 2005.

(51) Int. Cl. A61K 35/20; A61K 31/22; A61K 31/198; A61K 31/205; A61K 31/736

(52) U.S. Cl. 424/535; 514/546; 514/554; 514/54; 514/561; 514/563

(57) ABSTRACT

Methods and compositions for the purposes muscle growth, recovery and/or strength are set forth. The compositions may include, but are not limited to, creatine monohydrate, glutamine alpha-ketoglutarate, glutamine, glutamine peptides, L-glutamine, L-leucine, L-valine, L-isoleucine and guar gum. The supplemental dietary compositions may be suitable for persons seeking to improve strength and recovery for exercise while supporting muscle growth.

### RELATED APPLICATIONS

The application is related to and claims benefit of priority to U.S. Provisional Patent Application Ser. No. 60/657,680, entitled “Supplemental Dietary Composition for Supporting Muscle Growth, Recovery and Strength,” filed Mar. 1, 2005, the disclosure of which is hereby fully incorporated by reference.

### FIELD OF THE INVENTION

The present invention relates to a supplemental dietary composition including whey protein concentrate, the supplemental dietary composition supporting muscle growth, recovery, and/or strength, and to a method of promoting same by consuming the supplemental dietary composition. Moreover the supplemental dietary composition can be easily mixed with a polar solvent using a spoon.

### SUMMARY OF THE INVENTION

The present invention provides for a supplemental dietary composition including whey protein concentrate, providing di-, tri-, oligo-, and polypeptides, combined with a blend of, e.g., creatine monohydrate, glutamine alpha-ketoglutarate, glutamine peptides, L-glutamine, L-leucine, L-valine, L-isoleucine, and guar gum, wherein the supplemental dietary composition supports muscle growth, recovery, and/or strength. Furthermore, owing to the inclusion of guar gum as an emulsifier, the supplemental dietary composition can be easily mixed with a polar solvent using a spoon as opposed to the traditional shaker or blender methods.

The present invention also provides, by the consumption of the supplemental composition, a method of supporting muscle growth, recovery, and/or strength as well as providing an excellent source of protein.

### DETAILED DESCRIPTION OF THE INVENTION

The present invention, according to various embodiments thereof, is directed to a supplemental dietary composition that includes whey protein concentrate, providing di-, tri-, oligo-, and polypeptides. According to various embodiments of the present invention, the supplemental dietary composition may also include a blend of, e.g. creatine monohydrate, glutamine alpha-ketoglutarate, glutamine peptides, L-glutamine, L-leucine, L-valine, L-isoleucine, and guar gum. The supplemental dietary composition according to this invention provides a method of supporting muscle growth, recovery, and/or strength as well as providing an excellent source of protein.

According to various embodiments of the present invention, the supplemental dietary composition may be consumed in any form. For instance, the dosage form of the supplemental dietary composition may be provided as, e.g., a powder beverage mix, a liquid beverage, a ready-to-eat bar or drink product, a capsule, a tablet, a caplet, or as a dietary gel. The most preferred dosage form is a powder beverage mix.

Furthermore, the dosage form of the supplemental dietary composition in accordance with this embodiment may be provided in accordance with customary processing techniques for herbal and/or dietary supplements in any of the forms mentioned above.

In each of two example embodiments of the present invention set forth hereinbelow, which are set forth in greater detail in Examples 1 and 2 below, a supplemental dietary composition is provided for supporting muscle growth, recovery, and/or strength. According to these embodiments, the supplemental composition promotes and supports muscle growth and provides an excellent source of protein. In this manner, the consumption of the supplemental composition provides for a method for promoting and supporting muscle growth, recovery, and/or strength and for providing an excellent source of protein. According to the example embodiments set forth below, the supplemental dietary composition may be provided and consumed in any form, e.g., a powder beverage mix, a liquid beverage, a ready-to-eat bar or drink product, a capsule, a tablet, a caplet, or as a dietary gel. However, the preferred form for consumption of the supplemental dietary composition in accordance with the example embodiments set forth below is the powder beverage mix.

Furthermore, the dosage form of the supplemental dietary composition, in accordance with the example embodiments set forth below, may be provided in accordance with customary processing techniques for herbal and/or dietary supplements, wherein the active ingredients are suitably processed into e.g., powder beverage mixes, liquid beverages, and/or ready-to-eat bars or drinks. Those of skill in the art will appreciate that the supplemental dietary composition set forth in the example embodiments below may contain a variety of, and any number of different, excipients.

Whey Protein Concentrate

Whey protein is a natural protein from cow's milk. It is a source of essential amino acids needed on a daily basis by the body. Whey proteins include whey isolates and whey concentrates. Whey protein isolates contain little to no fat, lactose or cholesterol.

Although the present invention is not to be limited by any theoretical explanation, it is believed that whey protein enhances muscle growth, assists in weight management, and improves immunity.

As used herein, a serving of the supplement comprises from about 0.01-100.0 grams. More preferably, a serving of the supplement comprises from about 1.0-50.0 g per serving. Most preferably, a serving of the supplement comprises about 20 g of whey protein per serving.

Infuse™

Infuse™ is a blend of creatine monohydrate, glutamine alpha-ketoglutarate, glutamine peptides, L-glutamine, L-leucine, L-valine, L-isoleucine, and guar gum. Infuse™ combines a high concentration of creatine with essential amino acids.

Although the present invention is not to be limited by any theoretical explanation, it is believed that Infuse™, as a unique creatine-amino acid blend which, with regular exercise, has the potential to assist in muscle growth, recovery, and/or strength.

As used herein, a serving of the supplement comprises from about 0.01-10.0 grams. More preferably, a serving of the supplement comprises from about 1.0-2.5 g per serving. Most preferably, a serving of the supplement comprises about 1.3 g of Infuse™ per serving.

Creatine

Creatine refers to the chemical compound N-methyl-N-guanyl glycine, CAS Registry No. 57-00-1, also known as, (ÿ-methyl guanido) acetic acid, N-(aminoiminomethyl)-N-glycine, and methylglycocyamine, and Methylguanidoacetic acid, and N-Methyl-N-guanylglycine. Creatine also includes derivatives of creatine such as esters, and amides, and salts, as well as other derivatives, including derivatives that become active upon metabolism.

Creatine is known to be present in the muscles of vertebrates. It is present in a phosphorylated and a non-phosphorylated form and it is involved in muscular contraction and the development of fatigue. Creatine is produced naturally by the body, but can also be obtained from animal foods.

Although the present invention is not to be limited by any theoretical explanation, it is believed that creatine increase strength, energy and muscle size as well as cell volumization and hydration. It is believed that increasing the amount of creatine within muscle enhances muscular performance and the amount of work that can be done by the muscle. It has been widely reported that elevating the muscle total creatine store can enhance performance during high-intensity exercise.

Creatine and creatine derivatives are widely available from a number of commercial sources. The supplement preferably comprises creatine monohydrate.

Glutamine Peptides

Glutamine peptides are peptide-bound amino acids containing L-glutamine. Although the present invention is not to be bound by any theoretical explanation, it is believed that L-glutamine depletion occurs as a result of metabolic stress. Glutamine peptides replenish this amino acid and build lean muscle tissue, support protein synthesis and strengthen the immune system.

Glutamine alpha-ketoglutarate

Glutamine alpha-ketoglutarate is a glutamine precursor. Although the present invention is not bound by any theoretical explanation, it is believed that glutamine alpha-ketoglutarate efficiently bypasses the gut and has the ability to enter the muscle cell for superior absorption and cell volumization.

L-glutamine

L-glutamine is a non-essential amino acid. L-glutamine is also known as 2-aminoglutaramic acid, levoglutamide, (S)-2, 5-diamino-5-oxopentaenoic acid and glutamic acid 5-amide. Although the present invention is not to be bound by any theoretical explanation, it is believed that L-glutamine aids in muscle recovery, improves endurance, reduces fatigue, and strengthens the immune system.

L-leucine

L-leucine is an essential amino acid. L-leucine is also known as 2-amino4 methylvaleric acid, alpha-aminoisocaproic acid and (S)-2-amino4-methylpentanoic acid. Although the present invention is not to be bound by any theoretical explanation, it is believed that L-leucine is involved in muscle synthesis, as well as helping to promote healing of muscle, bone, and skin tissue. L-leucine is a three branched-chain amino acid which allows it to be used as an energy source by muscle tissue.

L-valine

L-valine is an essential amino acid. It is also known as 2-aminoisovaleric acid, 2-amino-3-methylbutyric acid, alpha-aminoisovaleric acid and (S)-2-amino-3-methylbutanoic acid. Although the present invention is not to be bound by any theoretical explanation, it is believed that L-valine aids in muscle metabolism, tissue repair, and helps maintain proper nitrogen balance in the body. L-valine is a three branched-chain amino acid which allows it to be used as an energy source by muscle tissue.

L-isoleucine

L-isoleucine is an essential amino acid. It is also known as 2-amino-3-methylvaleric acid, alpha-amino-beta-methylvaleric acid and (2S, 3S)-2-amino-3-methylpentanoic acid. Although the present invention is not to be bound by any theoretical explanation, it is believed that L-isoleucine increases energy levels, enhances endurance, and is necessary for hemoglobin formation.

Guar Gum

Guar gum is used as a thickener, stabilizer, and emulsifier in commercial food processing. It hydrates fairly rapidly in cold water to reduce the viscosity of highly viscous solutions. It is often found in salad dressings, sauces, milk products, and baking mixes. Guar gum allows the mixture of a powder beverage mix and a liquid to form an even consistency.

When the supplemental dietary composition is supplied as a dry beverage powder, preferably, 1-2 scoops of powder beverage mix should be mixed with 4-8 ounces of cold water or skim milk using a spoon or shaker. Preferably, two servings of the supplemental dietary composition will be consumed up to three times daily, for a minimum of six weeks.

Although the following examples illustrate the practice of the present invention in two of its embodiments, the examples should not be construed as limiting the scope of the invention. Other embodiments will be apparent to one skilled in the art from consideration of the specification of the following examples.

### EXAMPLES

### Example 1

### Chocolate Flavor

A serving of the supplemental dietary composition may include, but is not limited to, the following ingredients. Suggested directions are also provided.

| # | Ingredients | | g/serving | Formula % |
|---|---|---|---|---|
| 1 | WPC-80 DB, INSTANT | | 22.8664 | 84.690 |
| 2 | Protein, WPI-97 (IE) | | 1.1000 | 4.074 |
| 3 | Flavor, Chocolate | | 0.5650 | 2.093 |
| 4 | Protein, WPC-80 Part Hydrolyzed | | 0.2000 | 0.741 |
| 5 | Fat, Borage oil (dry) | | 0.0300 | 0.111 |
| 6 | Flax, oil powder, 30-36% | | 0.0300 | 0.111 |
| 7 | Sweetener, hi intensity, Sucralose | | 0.0230 | 0.085 |
| 8 | Sweetener, hi intensity, Acesulfame K | | 0.0230 | 0.085 |
| 9 | Infuse ™ | Creatine | 1.0000 | 3.704 |
| | | Monohydrate | | |
| | | Guar Gum | 0.2830 | 1.048 |
| | | L-leucine | 0.0016 | 0.006 |
| | | L-Valine | 0.0016 | 0.006 |
| 10 | | L-Isoleucine | 0.0016 | 0.006 |
| 11 | | L-Glutamine | 0.0016 | 0.006 |
| 12 | | Glutamine | 0.0016 | 0.006 |
| | | peptides | | |
| 13 | | Glutamine | 0.0016 | 0.006 |
| | | Alpha- | | |
| | | ketoglutarate | | |
| 14 | Natural Dried Cream Extract | | 0.0950 | 0.352 |
| 15 | Vanilla Flavor | | 0.1080 | 0.400 |
| 16 | Premium Cocoa Powder | | 1.1600 | 4.295 |
| 17 | French Vanilla Flavor | | 0.0680 | 0.252 |
| | | TOTAL | 27.000 | 100.000% |

Add 1 serving (27 g) of the dietary supplement to 4 oz. of cold water or skim milk, mix with spoon and consume immediately. For maximum muscle growth, consume 2 servings of the supplemental dietary composition up to 3 times daily for a minimum of 6 weeks.

### Example 2

### Vanilla Flavor

A serving of the supplemental dietary composition may include, but is not limited to, the following ingredients. Suggested directions are also provided.

| # | Ingredients | | g/serving | Formula % |
|---|---|---|---|---|
| 1 | WPC-80 DB, INSTANT | | 24.1254 | 89.353 |
| 2 | Protein, WPI-97 (IE) | | 1.1000 | 4.074 |
| 3 | Flavor, Vanilla | | 0.5650 | 2.093 |
| 4 | Protein, WPC-80 Part Hydrolyzed | | 0.2000 | 0.741 |
| 5 | Fat, Borage oil (dry) | | 0.0300 | 0.111 |
| 6 | Flax, oil powder, 30-36% | | 0.0300 | 0.111 |
| 7 | Sweetener, hi intensity, Sucralose | | 0.0230 | 0.085 |
| 8 | Sweetener, hi intensity, Acesulfame K | | 0.0230 | 0.085 |
| 9 | Infuse ™ | Creatine | 1.0000 | 3.704 |
| | | Monohydrate | | |
| | | Guar Gum | 0.2830 | 1.048 |
| | | L-leucine | 0.0016 | 0.006 |
| | | L-Valine | 0.0016 | 0.006 |
| 10 | | L-Isoleucine | 0.0016 | 0.006 |
| 11 | | L-Glutamine | 0.0016 | 0.006 |
| 12 | | Glutamine | 0.0016 | 0.006 |
| | | peptides | | |
| 13 | | Glutamine | 0.0016 | 0.006 |
| | | Alpha- | | |
| | | ketoglutarate | | |
| 14 | Vanilla Flavor | | 0.1080 | 0.110 |
| 15 | French Vanilla Flavor | | 0.6800 | 0.252 |
| | | TOTAL | 27.000 | 100.000% |

Add 1 serving (27 g) of the dietary supplement to 4 oz. of cold water or skim milk, mix with spoon and consume immediately. For maximum muscle growth, consume 2 servings of the supplemental dietary composition up to 3 times daily for a minimum of 6 weeks.

What is claimed:

1. A supplemental dietary composition for at least one of improving recovery from exercise, improving strength, and supporting muscle growth, the composition comprising:
creatine and/or salts and/or esters thereof;
whey protein including at least one of whey protein concentrate, whey protein isolate, and partially hydrolyzed whey protein; and
a blend that includes one or more of creatine monohydrate, glutamine alpha-ketoglutarate, glutamine peptides, L-glutamine, L-leucine, L-valine, L-isoleucine, and guar gum.

2. The supplement dietary composition of claim 1, wherein the creatine is creatine monohydrate.

3. The supplemental dietary composition of claim 1, wherein the creatine is approximately 4% of a serving.

4. The supplemental dietary composition of claim 1, wherein the whey protein is provided as a mixture of whey protein concentrate, whey protein isolate, and partially hydrolyzed whey protein.

5. The supplemental dietary composition of claim 1, wherein the total amount of whey protein provided is approximately 75% of a serving.

6. The supplement dietary composition of claim 1, wherein the whey protein concentrate provides di-, tri-, oligo-, and polypeptides.

7. The supplemental composition of claim 1, wherein the blend includes comprises L-leucine, L-valine, L-isoleucine, and L-glutamine, glutamine peptides, glutamine alpha-ketoglutarate and guar gum.

8. The supplemental dietary composition of claim 1, further comprising L-leucine, L-valine, L-isoleucine, and L-glutamine.

9. The supplemental dietary composition of claim 1, further comprising glutamine peptides.

10. The supplemental dietary composition of claim 1, further comprising glutamine alpha-ketoglutarate.

11. A supplemental dietary composition comprising:
whey proteins;
borage oil;
flax seed oil powder;
sucralose;
acesulfame K; and
a blend that includes one or more of creatine monohydrate, glutamine alpha-ketoglutarate, glutamine peptides, L-glutamine, L-leucine, L-valine, L-isoleucine, and guar gum,
wherein the supplemental dietary composition can be easily mixed with a polar solvent employing a spoon.

12. A supplemental dietary composition comprising:
whey proteins;
borage oil;
flax seed oil powder;
sucralose;
acesulfame K; and
a blend that includes one or more of creatine monohydrate, glutamine alpha-ketoglutarate, glutamine peptides, L-glutamine, L-leucine, L-valine, L-isoleucine, and guar gum,
wherein the supplemental dietary composition can be easily mixed with a polar solvent employing a shaker.

13. The supplemental dietary composition of claim 1, wherein the whey protein and creatine are present in an amount effective to at least one of provide improved recovery from exercise, improve strength, and/or support muscle growth and development.

14. A method for one of improved recovery from exercise, improved strength, and/or supporting muscle growth and development, the method comprising the steps of:
ingesting a supplemental dietary composition that includes:
creatine;
whey protein; and
a blend that includes one or more of creatine monohydrate, glutamine alpha-ketoglutarate, glutamine peptides, L-glutamine, L-leucine, L-valine, L-isoleucine, and guar gum.

15. The method of claim 14, wherein the creatine is creatine monohydrate.

16. The method of claim 14, wherein the creatine is approximately 4% of a serving.

17. The method of claim 14, wherein the whey protein is provided as a mixture of whey protein concentrate, whey protein isolate, and partially hydrolyzed whey protein.

18. The method of claim 14, wherein the total amount of whey protein provided is approximately 75% of a serving.

19. The method of claim 14, wherein the whey protein concentrate provides di-, tri-, oligo-, and polypeptides.

20. The method of claim 14, wherein the blend includes L-leucine, L-valine, L-isoleucine, L-glutamine, glutamine peptides, glutamine alpha-ketoglutarate and guar gum.

21. The method of claim 14, further comprising L-leucine, L-valine, L-isoleucine, and L-glutamine.

22. The method of claim 14, further comprising glutamine peptides.

23. The method of claim 14, further comprising glutamine alpha-ketoglutarate.

24. The method of claim 14, wherein the whey protein and creatine are present in an amount effective to at least one of provide improved recovery from exercise, improve strength, and/or support muscle growth and development.

25. A method of mixing a supplemental dietary composition comprising whey proteins, borage oil, flax seed oil powder, sucralose, acesulfame K, and a blend that includes one or more of creatine monohydrate, glutamine alpha-ketoglutarate, glutamine peptides, L-glutamine, L-leucine, L-valine, L-isoleucine, and guar gum, wherein the method includes the step of mixing the supplemental dietary composition with a polar solvent employing a spoon.

26. A method of mixing a supplemental dietary composition comprising whey proteins, borage oil, flax seed oil powder, sucralose, acesulfame K, and a blend that includes one or more of creatine monohydrate, glutamine alpha-ketoglutarate, glutamine peptides, L-glutamine, L-leucine, L-valine, L-isoleucine, and guar gum, wherein the method includes the step of mixing the supplemental dietary composition with a polar solvent employing a shaker.
